(19) United States
(12) Patent Application Publication
(10) Pub. No.: US 2017/0120086 A1
(43) Pub. Date: May 4, 2017

(54) Nose apparatus

(71) Applicant: Mariusz Androsiuk, Sopot (PL)

(72) Inventor: Mariusz Androsiuk, Sopot (PL)

(73) Assignee: andervision sp. z o.o., Gdynia (PL)

(21) Appl. No.: 15/334,323

(22) Filed: Oct. 26, 2016

(30) Foreign Application Priority Data
Oct. 31, 2015 (PL) P.414634

(51) Int. Cl. A62B 23/06; A61M 31/00; A62B 9/04

(52) U.S. Cl. CPC A62B 23/06; A62B 9/04; A61M 31/00

(57) ABSTRACT

Nose apparatus having two flat oval replaceable filtering inserts connected to each other, characterised in that it has an external flexible carrying frame (1) in the shape resembling reversed letter V matching and adjusted to be fixed on the nasal bridge, to the bottom ends of which are fixed horizontal clamps (3) of the filtering inserts (4) and vertical elements (2) adjusted to be placed inside the nasal openings, where the clamps (3) with the filtering inserts (4) fit tight on the outside of the nasal openings, and the bottom ends of the flexible carrying frame (1) together with the vertical elements (2) form clasps on the nostrils.

The invention concerns a nose apparatus. It is used to protect human upper respiratory tract against air pollutants, especially the PM 10 and PM 2.5 particulate matter. It enables fixing a structure with replaceable filters on the nose, where the filters serve as a barrier for the harmful particles in the air. The nose apparatus is a personal health protection device which pertains to the reduction of illness incidence and the number of deaths caused in the society by infections of the upper respiratory tract. The proposed structure of the device enables its broad application when performing any type of physical activity, thus offering an effective technical solution protecting the humans from inhaling polluted air through the nose.

Respiration is the fundamental activity which keeps us alive. Every minute we supply 6-7 liters of air to the lungs, which gives ca. 14 thousand liters in 24 hours. Cleaning the air supplied to the lungs is one of the important functions of breathing through the nose. Approximately 90% of the pollutant particles larger than 10 mm in diameter get filtered out. With the diameter below 4 mm only 50% of particles are filtered. The smaller the pollutant particle, the easier it gets to our lungs.

According to World Health Organization (WHO), in 21st century more than two million people die each year because of inhaling polluted air, both fresh air outside, and the air filling the living spaces, particularly when close to hearths. Inhalation of particulate matter, particles below 10 micrometers in particular, is very dangerous and allows the matter to get readily from the human respiratory tract to the blood system. This causes a number of diseases related to damages to the heart, lung cancer, or asthma.

There are still many places around the world where the air is polluted and the maximum levels get exceeded several times or even a dozen-or-so times. The level of pollution has a direct impact on health of the people living there.

Known are different structures of personal filters, such as a diaphragm or barrier for polluted air. The best known method of protecting the upper respiratory tract consists in the use of different kinds of face masks, which are obviously conspicuous. While advantages of the solutions of the type are obvious, particularly in industrial or medical surroundings, their use in everyday life in the society during different activities is arguable, even though in some countries of the Far East, Japan in particular, wearing filtering masks on the face is no longer seen as an extravagance but as a necessary measure of personal protection of the respiratory tract against air pollutants, as well as a protection of other persons against contracting an infection from those already infected.

Other technical solutions among systems which enable filtering polluted air inhaled through the nose are based on placing the filtering layer either outside the nasal vestibule by e.g. sticking it to the nostrils, or inside the vestibules by pushing two filters therein. The filters are connected to each other with a connector running under the nasal septum. The solution is hardly visible for incidental observers, which is an unquestionable advantage when used in public life. An obvious disadvantage, however, is the discomfort caused by the filters used inside the nasal vestibules which distends them slightly since it is necessary for the filters to fill the vestibules tightly and wedge in place inside. The discomfort is reported as the major reservation in the tests related to the assessment of the comfort of using the so-called nasal filtering plugs. A slight discomfort can be felt all the time they are being worn. In general, the solution can be found in two variants. In the first variant, the filters which are inserted into the nasal vestibules are usually conical in shape, which causes much more discomfort in use because of the relatively large weight of the elements, and although they improve the degree to which the air is filtered, at the same time they increase the air flow resistance. In the second variant the filters are flat and less invasive. Some are more flexible and adjust better to the movements of nostrils while in use. Both variants, however, are scarred with the problem of filling the nasal vestibules tightly so as to prevent inhaling non-filtered air through the slots which form when the nose moves, there is also the previously-mentioned problem of discomfort caused by the distended nasal vestibules. Found in both variants are solutions composed of a number of elements, including the option of replacing the filtering inserts, which is an obvious advantage on the one hand, but on the other hand it also causes a number of problems related to the increased amount of the material which should fit in the nasal vestibules or under the nose, as well as to the tightness inside the vestibules, and the increased weight to be supported in use.

Known from international patent application No. WO2005/120645 is a nose filtering device fitted with flexible elements to support the filtering material basically circular in shape, and a positioning element which links the supporting elements, placed under the nasal septum. The supporting elements with the filtering material are placed inside the nasal openings. Disclosed are different variants of construction of the filtering material and its supporting elements.

Known from patent application published under No. CN201482019 is a device for purification of air in the nostrils, having filtering screens in flexible rims connected with a clasping element placed under the nasal septum.

Also known from patent application No. U52012/0279504 is a nose filter containing flat filtering elements which cover the nasal openings directly. The structure contains dilating elements which adjust to the nasal passage.

Disclosed in patent application No. WO2014/056501 is a nose filter having a frame composed of the left and right parts the dimensions of which are selected so that they can be placed inside the left and right nasal opening. Optionally, the parts can be connected with a U-shaped bridge running under the nasal septum. Described are the shapes and properties of individual sections of the frame, in particular of its front and back sections, as well as the connections between the sections and their shapes.

All solutions listed above offer many advantages, but are not free from minor or major disadvantages which reduce the probability they will be actually used by the potential clients. The purpose of the new technical solution in this field is to propose a structure which will be acceptable and attractive for the clients in everyday use. Such technical solution should meet a number of assumptions. First of all, it should be light and comfortable to wear, and its method of fixing minimally invasive. Secondly, the filtering inserts should contact the nostrils tightly while enabling free movements of the nose without too much interference into its inner space. Thirdly, the solution should enable easy replacement of the filtering inserts without the need to replace the entire device. In addition, it should enable personalization of each set with the possibility of selecting the perfect size and matching the components for their colors and external materials so that the entire set forms a product attractive visually and comfortable to wear. The purpose is to build the image of the filtering apparatus as a desirable sport accessory for active people aware of the hazards resulting from the inhalation of polluted air and the related allergies and diseases of the upper respiratory tract.

Nose apparatus having two flat oval replaceable filtering inserts connected to each other, characterised in the invention that it has an external flexible carrying frame in the shape resembling reversed letter V matching and adjusted to be fixed on the nasal bridge, to the bottom ends of which are fixed horizontal clamps of the filtering inserts and vertical elements adjusted to be placed inside the nasal openings. The clamps with the filtering inserts fit tight on the outside of the nasal openings, and the bottom ends of the flexible carrying frame together with the vertical elements form clasps on the nostrils.

In one variant, the vertical elements are formed by bending the bottom ends of the flexible carrying frame, where connected to the bent ends are the horizontal clamps.

The horizontal clamps are non-detachably connected to the vertical elements.

In the second variant, the horizontal clamps are detachably and rotatingly connected to the vertical elements.

Preferably the vertical elements take the form of cylindrical pegs non-detachably connected to the horizontal clamps.

The horizontal clamps are non-detachably connected to the bottom ends of the carrying frame.

Preferably the horizontal clamps are rotatingly and detachably connected to the bottom ends of the carrying frame.

Preferably the horizontal clamps have the shape of incomplete rings open on the side opposite to the vertical elements, and the filtering inserts have two ring flanges with clearances on the side of the vertical elements, between which the filtering material is placed, where the horizontal clamps are inserted in between the flanges of the filtering inserts.

In the next variant, there is a groove along the circumference of at least one horizontal clamp, where fitted in the groove, between the top horizontal wall of the clamp and the top flange of the filtering insert is an additional insert featuring indentations filled with an additional substance, and the top flange of the filtering insert and the top horizontal wall of the clamp have pass-through openings corresponding to the indentations in the additional insert.

Aroma is used as the additional substance.

A medical substance is used as the additional substance.

Preferably, members of the apparatus, namely: the carrying frame, vertical elements, clamps, ring flanges of the filtering insert and additional aromatic inserts are made of a flexible plastic material intended for use in medical products, fit for contact with human skin and enabling comfortable use of the nose apparatus, especially when the fascial muscle, nose in particular, move. The only components which get inside the nasal vestibules are small vertical elements which together with the carrying frame form clasps on the nostrils. The replaceable filtering inserts are selected individually to match the size and shape of the nasal openings of the user, so that the size of the inserts is larger than the maximum diameters of the nasal openings resulting from the muscular movements of the face, while fitting therein tightly and comfortably. Moreover, the soft material, e.g. silicone of an appropriate type, used to make the ring flanges on the filtering inserts causes build-up of underpressure when inhaling through the nose, in effect of which the soft ring flanges on the filtering insert mounted loose on the nostril clasp are pressed tight, thus improving the tightness of passage of the outside air which should flow through the filtering material only. Another advantage of the solution is the profiling of the carrying frame which matches the shape of the nose, and distribution of the small weight of the nose apparatus not only on the two nostril clasps, but also on the arched top part of the carrying frame through which it is transferred on the nasal septum. This improves the stability of the entire apparatus on the nose and its safety when ejecting air rapidly from the lungs e.g. in sneezing. It is also possible to adjust the carrying frame to different sizes and shapes of the noses by bending it to fit or replacing the profile to change the length, as well as to select the color and texture according to the taste of the user. Yet another advantage of the described solution consists in the use of filtering inserts which enable their easy replacement and fitting in the clamps of the nose apparatus, as well as the possibility to use additional inserts with indentations filled with an aroma or medical substance. The location of the indentations and of the pass-through openings along the edge of the clamps and not within the space of the filtering material does not inhibit the air flow to the lungs.

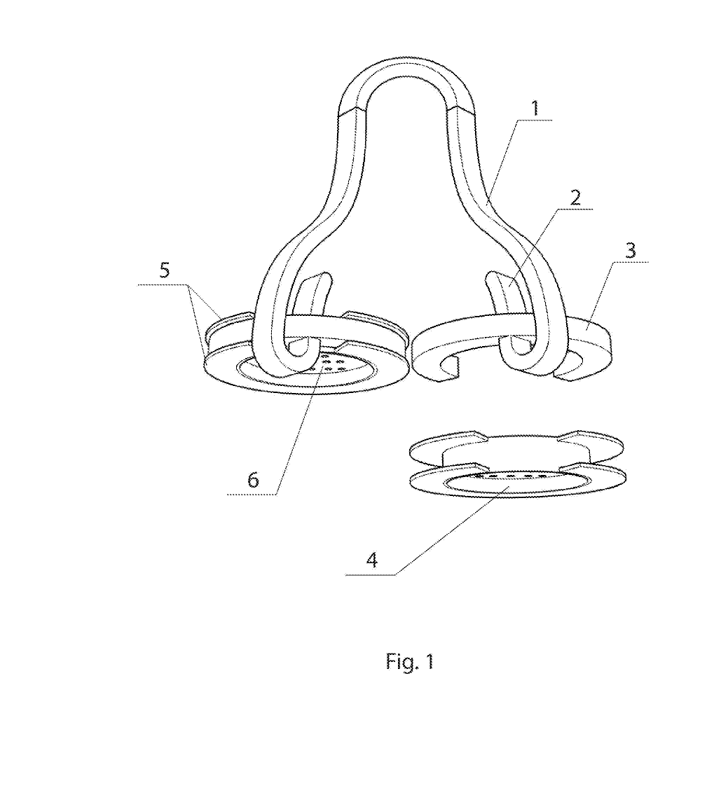

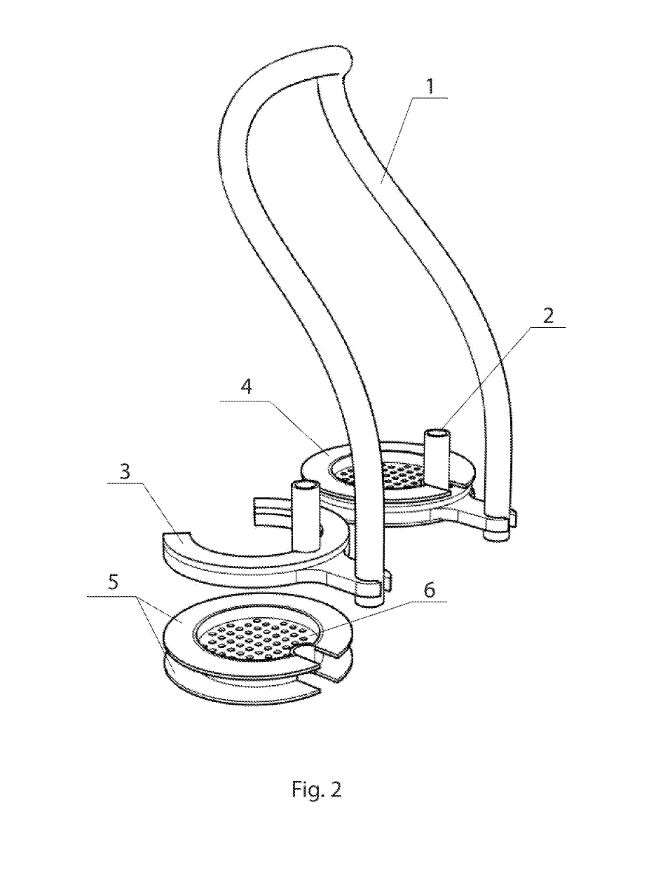

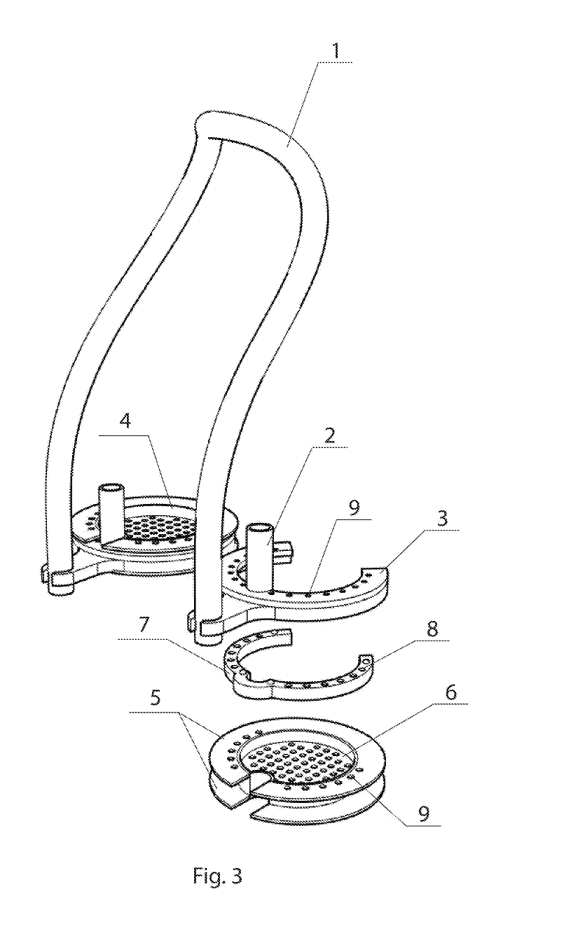

An exemplary embodiment of the nose apparatus is illustrated on a drawing, where FIG. 1 depicts the nose apparatus in the first variant with a single filtering insert removed, FIG. 2 shows the nose apparatus in the second variant with a single filtering insert removed, FIG. 3 depicts the nose apparatus in the third variant with a single filtering insert removed and with an additional insert.

In the first exemplary embodiment of the invention shown on FIG. 1 the nose apparatus has a flexible carrying frame 1 in the shape resembling reversed letter V rounded on the top, with its bottom ends bent to form short vertical elements 2. Non-detachably connected to the vertical elements 2 are horizontal clamps 3. The shape of the carrying frame 1 is adjusted to the shape of the nose, where the frame and the vertical elements are formed of flexible plastic profile rectangular in cross section. The clamps are made of plastic and have the shape of incomplete rings open on the side opposite to the vertical elements 2, adjusted to the shape of the filtering inserts 4. Each of the two filtering inserts 4 has two ring flanges 5, inside which the filtering material 6 is placed. Inserted in between the flanges 5 are clamps 3. Along the circumference of the ring flanges 5 of the filtering inserts 4 there are clearances for vertical elements 2. Once the filtering inserts 4 are connected to the clamps 3, the nose apparatus is placed on the bridge of the user's nose so that the nostrils are between the vertical elements 2 and the bottom ends of the carrying frame 1, and the clamps 3 with the filtering inserts are under the nasal openings. The apparatus is fixed on the user's nose and taken off by putting the index fingers on the arch of the carrying frame 1 and simultaneous tilting the vertical elements 2 with two thumbs put on the bottom ends of the carrying frame 1. The distance between the ends of the vertical elements 2 and the carrying frame 1 increases and the clasp on the nostrils is released. The filtering inserts 4 consist of two ring flanges 5 connected together, and the filtering material 6. The ring flanges 5 are made of flexible plastic (silicone). Used as the filtering material 6 can be either woven materials, such as carbon cellulosic fabric, or non-woven materials of the ‘spunbond’ type, produced in the process of direct formation of continuous fibers from polymer granulates (polypropylene PP or combination of polypropylene and polyethylene PP/PE). The main function of the filtering material 6 is to effectively filter the air inhaled through the nose to clean it from particulate matters PM 2,5 and PM 10 which are hazardous to human health. The filtering material 6 is spread between the ring flanges 5 and permanently fixed thereto. The filtering insert 4 is characterized by its elasticity which allows pressing it slightly e.g. between the thumb and the index finger, and slipping in on the arms of clamp 3. The clearance in the ring flange 5 should be oriented so that it matches the point of connection between the clamp 3 and the vertical element 2. The filtering insert 4 is replaced by slipping it off the arms of clamp 3. In the second exemplary embodiment shown on FIG. 2 the nose apparatus has a flexible carrying frame 1 in the shape resembling reversed letter V to the bottom ends of which are detachably and rotatingly fixed horizontal clamps 3, integrated with which are vertical elements 2 in the form of cylindrical pegs, placed close to the bottom ends of the carrying frame 1. The ring-shaped clamps 3 have clearances on the side opposite to the vertical elements 2, which enable slipping the filtering inserts 4 in place, where the filtering insert 4 has two ring flanges 5 in between which the filtering material 6 is placed. Once the filtering inserts 4 are connected to the clamps 3, the nose apparatus is placed on the bridge of the user's nose so that the nostrils are between the vertical elements 2 and the bottom ends of the carrying frame 1, and the clamps 3 with the filtering inserts are under the nasal openings. The apparatus is fixed on the user's nose and taken off by putting an index finger on the arch of the carrying frame 1 and simultaneous tilting the vertical elements 2 with two thumbs put on the bottom ends of the carrying frame 1. The distance between the ends of the vertical elements 2 and the carrying frame 1 increases and the clasp on the nostrils is released. The filtering inserts 4 consist of two ring flanges 5 connected together, and the filtering material 6. The ring flanges 5 are made of flexible plastic (silicone). Used as the filtering material 6 can be either woven materials, such as carbon cellulosic fabric, or non-woven materials. The main function of the filtering material 6 is to effectively filter the air inhaled through the nose to clean it from particulate matters PM 2,5 and PM 10 which are hazardous to human health. The filtering material 6 is spread between the ring flanges 5 and permanently fixed thereto. The filtering insert 4 is characterized by its elasticity which allows pressing it slightly e.g. between the thumb and the index finger, and slipping in on the arms of clasp 3. The clearance in the ring flange 5 should accommodate the vertical element 2. The filtering insert 4 is replaced by slipping it off the arms of clamp 3.

Thanks to the rotational connection between the clamps 3 and the carrying frame 1 it is possible to adjust the position of clamps 3 with the filtering inserts 4 to the individual spacing of the nasal openings, and the detachable method of fixing the clamps 3 to the carrying frame 1 allows to fold the set and make it more handy in transport when the nose apparatus is not used. Moreover, the structure of the apparatus allows to replace the carrying frame 1 and match the nose apparatus to the specific user.

In the third variant as shown on FIG. 3 the nose apparatus is fitted with an extra insert 7 with indentations 8 intended to be filled with additional substance, an aroma in particular. In exemplary embodiment of the variant the nose apparatus has a flexible carrying frame 1 in the shape resembling reversed letter V to the bottom ends of which are detachably and rotatingly fixed horizontal clamps 3, integrated with which are vertical elements 2 in the form of cylindrical pegs, placed close to the bottom ends of the carrying frame 1. The ring-shaped clamps 3 have clearances on the side opposite to the vertical elements 2, which enable slipping the filtering insert 4 in place, where the filtering insert 4 has two ring flanges 5 in between which the filtering material 6 is placed. There is a groove along the circumference of the clamps 3 in which an additional insert 7 is fitted between the top vertical wall of the clamp 3 and the bottom flange of the filtering insert 4. The additional insert 7 takes the form of an open ring corresponding in shape to the ring of the clamp 3, and the indentations 8 are made in its top wall. The top ring flange 5 of the filtering ring 4 and the top vertical wall of clamp 3 have pass-through openings 9 above the indentations 8 filled with additional substance. In the process of production, the indentations on the additional insert 7 filled with additional substance can be covered with a sealing film which would be pierced by the ends of the pass-through openings 9 the moment the inserts are slipped into the clamps 3.

1. Nose apparatus having two flat oval replaceable filtering inserts connected to each other, characterised in that it has an external flexible carrying frame (1) in the shape resembling reversed letter V matching and adjusted to be fixed on the nasal bridge, to the bottom ends of which are fixed horizontal clamps (3) of the filtering inserts (4) and vertical elements (2) adjusted to be placed inside the nasal openings, where the clamps (3) with the filtering inserts (4) fit tight on the outside of the nasal openings, and the bottom ends of the flexible carrying frame (1) together with the vertical elements (2) form clasps on the nostrils.

2. The apparatus according to claim 1, characterised in that the vertical elements (2) are formed by bending the bottom ends of the flexible carrying frame, where connected to the bent ends are the horizontal clamps (3).

3. The apparatus according to claim 2, characterised in that the horizontal clamps (3) are non-detachably connected to the vertical elements (2).

4. The apparatus according to claim 2, characterised in that the horizontal clamps (3) are detachably and rotatingly connected to the vertical elements (2).

5. The apparatus according to claim 1, characterised in that the vertical elements (2) take the form of cylindrical pegs non-detachably connected to the horizontal clamps (3).

6. The apparatus according to claim 5, characterised in that the horizontal clamps (3) are non-detachably connected to the bottom ends of the carrying frame (1).

7. The apparatus according to claim 5, characterised in that the horizontal clamps (3) are rotatingly and detachably connected to the bottom ends of the carrying frame (1).

8. The apparatus according to claims 1-7, characterised in that the horizontal clamps (3) have the shape of incomplete rings open on the side opposite to the vertical elements (2), and the filtering inserts (4) have two ring flanges (5) with clearances on the side of the vertical elements (2), between which the filtering material (6) is placed, where the horizontal clamps (3) are inserted in between the flanges (5) of the filtering inserts (4).

9. The apparatus according to claim 8, characterised in that there is a groove along the circumference of at least one horizontal clamp (3), where fitted in the groove, between the top horizontal wall of the clamp (3) and the top flange (5) of the filtering insert (4) is an additional insert (7) featuring indentations (8) filled with an additional substance, and the top flange (5) of the filtering insert (4) and the top horizontal wall of the clamp (3) have pass-through openings (9) corresponding to the indentations (8) in the additional insert (7).

10. The apparatus according to claim 9, characterised in that aroma is used as the additional substance.

11. The apparatus according to claim 9, characterised in that a medical substance is used as the additional substance.
